(19) United States
(12) Patent Application Publication
(10) Pub. No.: US 2026/0334791 A1
(43) Pub. Date: Oct. 29, 2026

(54) DETECTION DEVICE

(71) Applicant: Japan Display Inc., Tokyo (JP)

(72) Inventor: Gen KOIDE, Tokyo (JP)

(21) Appl. No.: 19/678,552

(22) Filed: May 15, 2026

Related U.S. Application Data
(63) Continuation of application No. PCT/JP2024/040471, filed on Nov. 14, 2024.

(30) Foreign Application Priority Data
Nov. 21, 2023 (JP) 2023-197726

(51) Int. Cl. A61B 5/00

(52) U.S. Cl. CPC A61B 5/0059; A61B 5/6802; A61B 2562/043

(57) ABSTRACT

According to an aspect, a detection device includes: a first substrate having a notch between both ends in a first direction; a light source disposed in the notch; a terminal part provided at one end of the first substrate in the first direction; at least one first optical sensor and at least one second optical sensor provided to the first substrate. The first and second optical sensors are disposed so as to sandwich the notch in the first direction. The first and second optical sensors each comprise a lower electrode, a lower buffer layer, and an active layer. The first and second optical sensors each comprise an upper electrode provided on the active layer and supplied with a reference potential from the terminal part. The lower electrodes of the first and second optical sensors are electrically coupled to each other and are electrically coupled to the terminal part.

### CROSS-REFERENCE TO RELATED APPLICATION

This application claims the benefit of priority from Japanese Patent Application No. 2023-197726 filed on Nov. 21, 2023 and International Patent Application No. PCT/JP2024/040471 filed on Nov. 14, 2024, the entire contents of which are incorporated herein by reference.

### BACKGROUND

### 1. Technical Field

What is disclosed herein relates to a detection device.

### 2. Description of the Related Art

Optical sensors capable of detecting fingerprint patterns and vein patterns are known (for example, Japanese Patent Application Laid-open Publication No. 2009-32005). Examples of such optical sensors include a sensor with a plurality of photodiodes using an organic semiconductor material as an active layer. Light emitted from a light source is scattered and reflected by a detection target and enters each of the photodiodes. Thus, the photodiodes detect biometric information on the detection target.

When the photodiodes of such an optical sensor are arranged in an array, the light-receiving sensitivity of the optical sensor may be lowered because the total light-receiving area of the photodiodes is small.

For the foregoing reasons, there is a need for a detection device capable of improving accuracy of detection.

### SUMMARY

According to an aspect, a detection device includes: a first substrate having a notch between both ends in a first direction; a light source disposed in the notch of the first substrate; a terminal part provided at one end of the first substrate in the first direction; at least one first optical sensor provided to the first substrate; and at least one second optical sensor provided to the first substrate. The first optical sensor and the second optical sensor are disposed so as to sandwich the notch of the first substrate in the first direction. The first optical sensor and the second optical sensor each comprise a lower electrode, a lower buffer layer, and an active layer. The first optical sensor and the second optical sensor each comprise an upper electrode provided on the active layer and supplied with a reference potential from the terminal part. The lower electrode of the first optical sensor and the lower electrode of the second optical sensor are electrically coupled to each other and are electrically coupled to the terminal part.

### BRIEF DESCRIPTION OF THE DRAWINGS

FIG. 1 is a schematic view illustrating an exemplary external appearance when a state of a finger accommodated inside a detection device according to a first embodiment is viewed from a lateral side of a housing;

FIG. 2 is a schematic sectional view taken along II-II' illustrated in FIG. 1;

FIG. 3 is a developed view illustrating a development example of the detection device illustrated in FIG. 1;

FIG. 4 is a configuration diagram illustrating a configuration example of a first optical sensor and a second optical sensor illustrated in FIG. 3;

FIG. 5 is a schematic sectional view illustrating a multilayered configuration example of the optical sensor taken along V-V' illustrated in FIG. 4;

FIG. 6 is a schematic sectional view illustrating a multilayered configuration example of the optical sensor taken along VI-VI' illustrated in FIG. 4;

FIG. 7 is a circuit diagram illustrating a configuration example of a detection circuit according to an embodiment;

FIG. 8 is a configuration diagram illustrating a configuration example of a first optical sensor and a second optical sensor according to a comparative example;

FIG. 9 is a configuration diagram illustrating a configuration example of a first optical sensor and a second optical sensor according to a second embodiment; and

FIG. 10 is a schematic sectional view illustrating a configuration example of a detection device according to the second embodiment.

### DETAILED DESCRIPTION

The following describes modes (embodiments) for carrying out the present disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

In the present specification and claims, in expressing an aspect of disposing another structure on or above a certain structure, a case of simply expressing "on" includes both a case of disposing the other structure immediately on the certain structure so as to contact the certain structure and a case of disposing the other structure above the certain structure with still another structure interposed therebetween, unless otherwise specified.

### FIRST EMBODIMENT

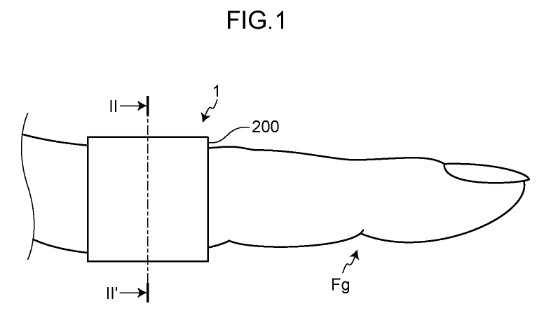

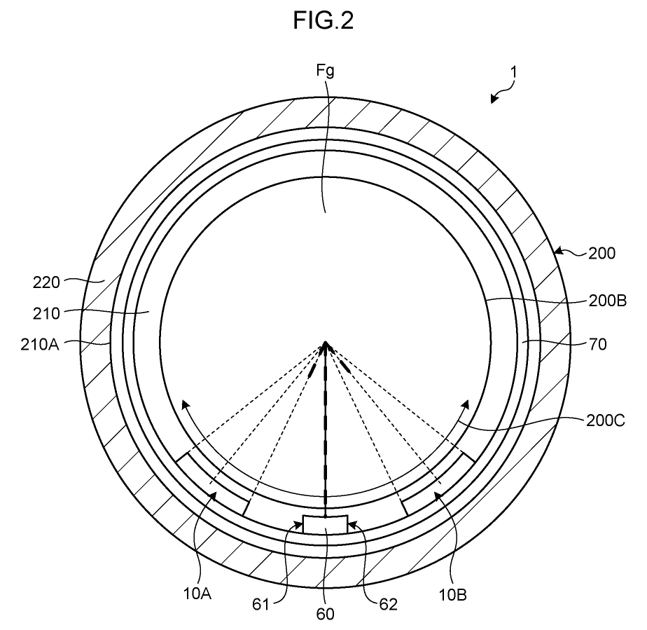

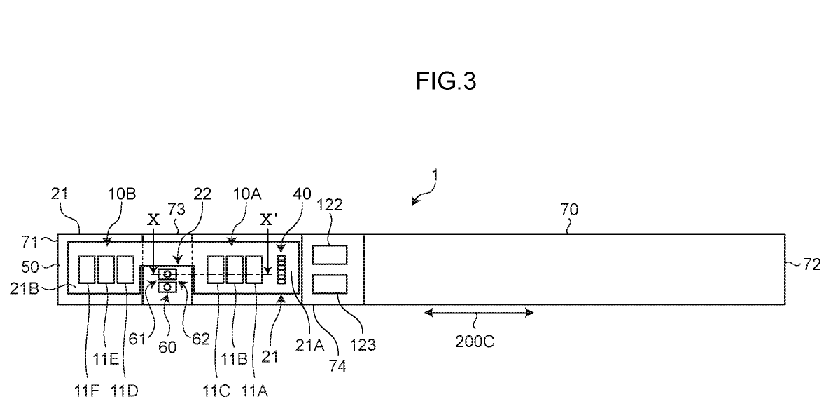

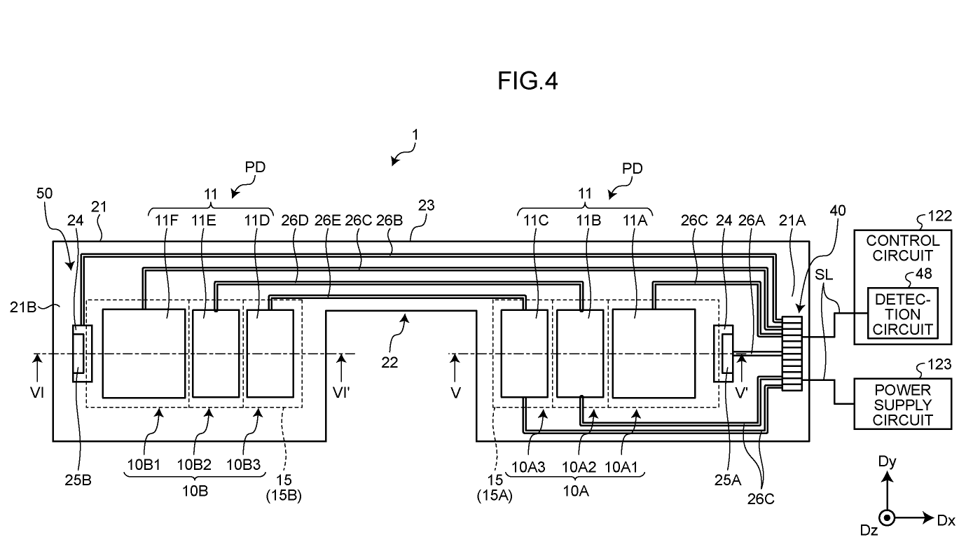

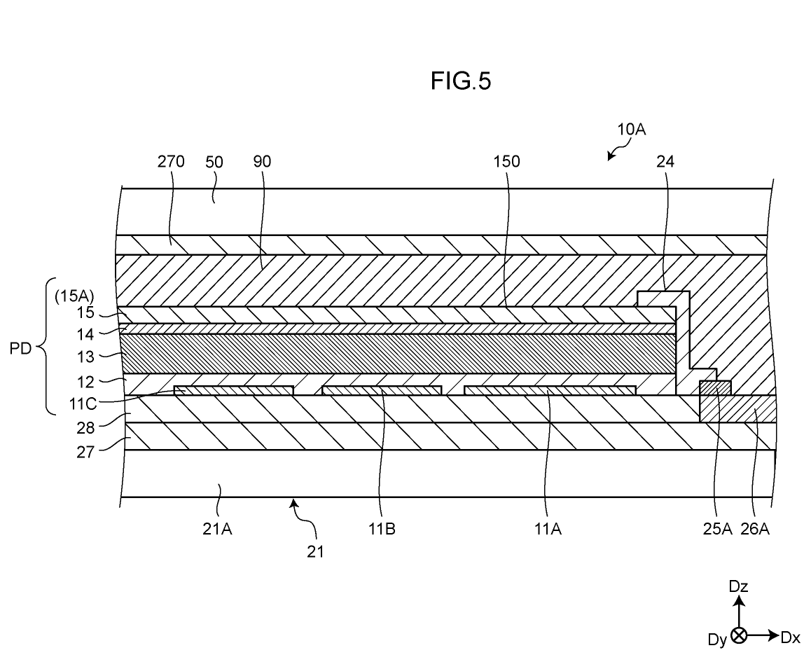

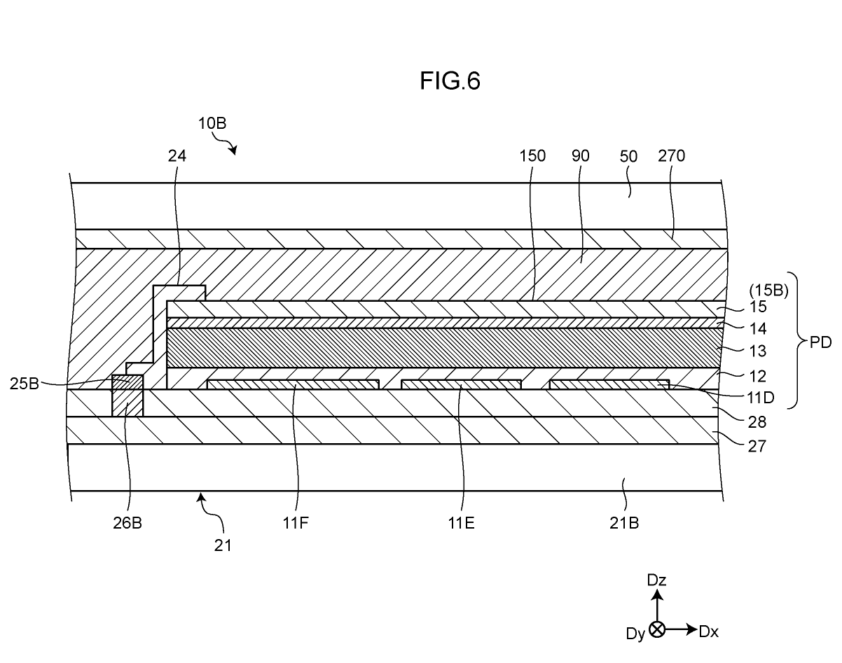

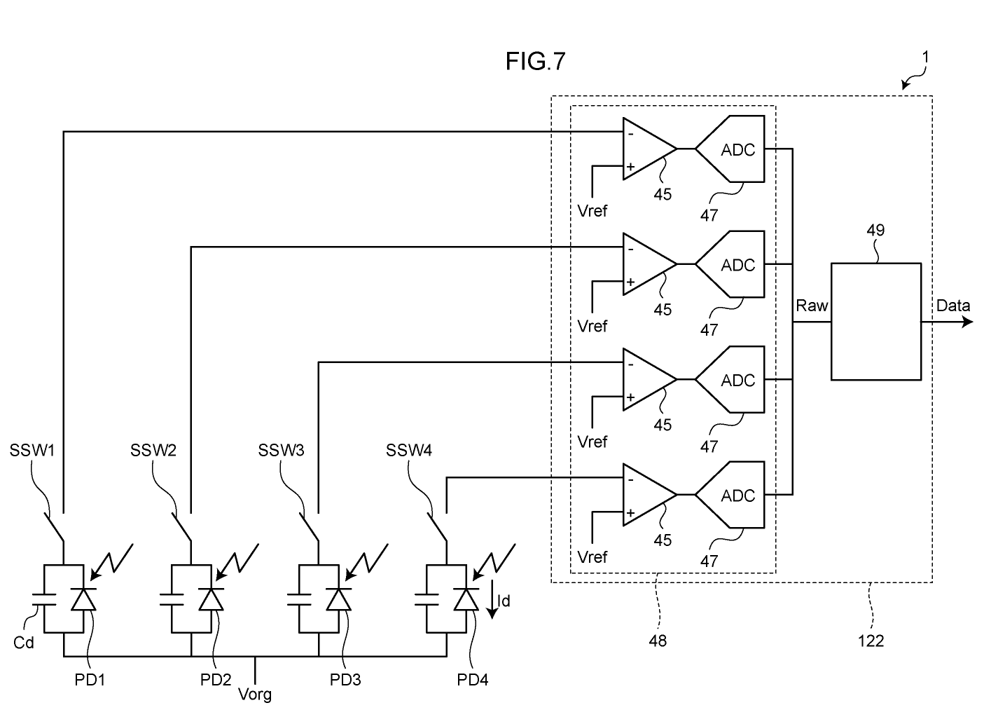

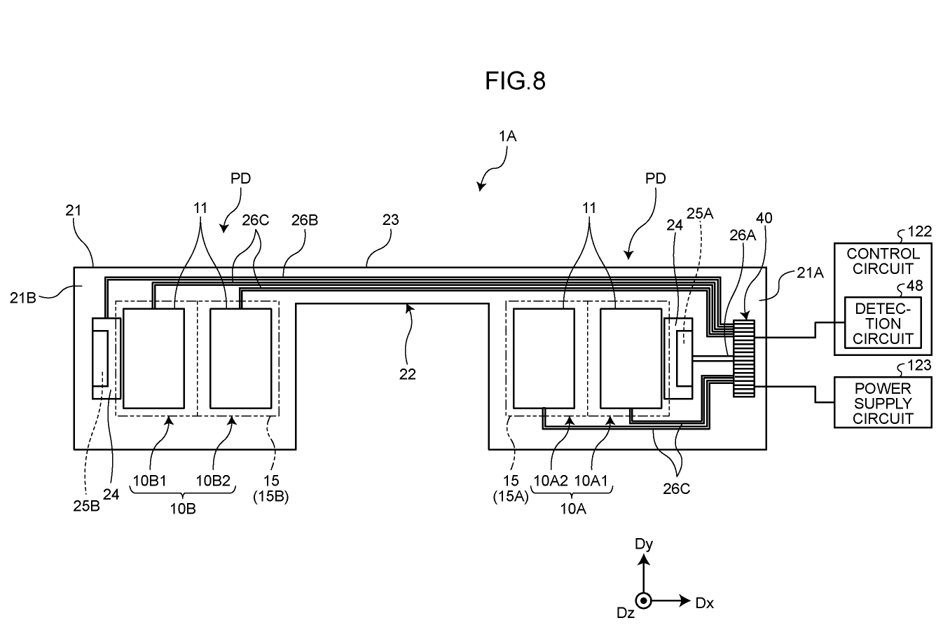

FIG. 1 is a schematic view illustrating an exemplary external appearance when a state of a finger accommodated inside a detection device according to a first embodiment of the present disclosure is viewed from a lateral side of a housing. FIG. 2 is a schematic sectional view of a section taken along II-II' illustrated in FIG. 1. FIG. 3 is a developed view illustrating a development example of the detection device illustrated in FIG. 1. FIG. 4 is a configuration diagram illustrating a configuration example of a first optical sensor and a second optical sensor illustrated in FIG. 3. FIG. 5 is a schematic sectional view illustrating a multilayered configuration example of the optical sensor taken along V-V' illustrated in FIG. 4. FIG. 6 is a schematic sectional view illustrating a multilayered configuration example of the optical sensor taken along VI-VI' illustrated in FIG. 4. FIG. 7 is a circuit diagram illustrating a configuration example of a detection circuit according to an embodiment. FIG. 8 is a configuration diagram illustrating a configuration example of a first optical sensor and a second optical sensor according to a comparative example.

A detection device 1 illustrated in FIG. 1 is a finger ring-shaped device that can be worn on and removed from a human body and is worn on a finger Fg of the human body. Examples of the finger Fg include a thumb, an index finger, a middle finger, a ring finger, and a little finger. The human body is a person to be authenticated whose identity is to be verified by the detection device 1. The detection device 1 can detect biometric information on a living body from the finger Fg wearing the detection device 1. The finger Fg is an example of a measurement target. The measurement target is the living body or a part of the living body to be measured. The detection device 1 is formed as a finger ring or a wristband so as to be easily carried by a user. In the following description, the detection device 1 is assumed to be used as a finger ring.

As illustrated in FIG. 2, the detection device 1 includes a housing 200, a light source 60, at least one first optical sensor 10A, at least one second optical sensor 10B, and a flexible printed circuit board 70. The detection device 1 is a device that includes a battery (not illustrated) inside the housing 200 and is operated by power of the battery.

The housing 200 is a wearable member that is formed in a ring shape (annular shape) wearable on the finger Fg, and is worn on the living body. In the example illustrated in FIG. 2, the housing 200 includes a first housing 210 and a second housing 220. The first housing 210 is integrated with the second housing 220 to form the housing 200 into the ring shape. The first housing 210 is a member that contacts the human body on which the housing 200 is worn. The first housing 210 accommodates therein the light source 60, the first optical sensor 10A, the second optical sensor 10B, and other components. The first housing 210 is formed into a ring shape using a housing material, such as a light-transmitting synthetic resin or silicone. The second housing 220 has a surface of the housing 200 that covers an outer peripheral surface 210A of the first housing 210. The second housing 220 is formed into a ring shape using a member of, for example, a metal or a non-light-transmitting synthetic resin. The first housing 210 of the housing 200 accommodates the flexible printed circuit board 70 on which the light source 60, the first optical sensor 10A, the second optical sensor 10B, and other components are fabricated. The flexible printed circuit board 70 is accommodated in the housing 200, for example, by being formed into a ring shape and, in that state, filling a periphery thereof with a filling material in a mold to form the housing 200.

As illustrated in FIG. 3, the flexible printed circuit board 70 has a deformable strip shape and is formed into a ring shape by coupling a first end 71 to a second end 72. The flexible printed circuit board 70 has a first fabrication region 73 and a second fabrication region 74. The first fabrication region 73 is a region on which the light source 60 and other components are fabricated. The second fabrication region 74 is a region on which a control circuit 122, a power supply circuit 123, and other components are fabricated. A first substrate 21 is provided on the flexible printed circuit board 70 so as to partially surround the light source 60 in the first fabrication region 73. The first substrate 21 is a sensor substrate provided with the first optical sensor 10A, the second optical sensor 10B, and other components. A second substrate 50 is provided on the first substrate 21. The flexible printed circuit board 70 electrically couples the light source 60, the first optical sensor 10A, the second optical sensor 10B, and other components, to the control circuit 122.

The first optical sensor 10A and the second optical sensor 10B according to the present embodiment are provided so as to sandwich the light source 60 in a circumferential direction 200C. In other words, the first optical sensor 10A, the light source 60, and the second optical sensor 10B of the detection device 1 are arranged in this order in the circumferential direction 200C. By being arranged so as to sandwich the light source 60 in the circumferential direction 200C, the first optical sensor 10A and the second optical sensor 10B can detect light emitted from the light source 60 over a wide area of the housing 200.

As illustrated in FIGS. 3 and 4, the detection device 1 further includes the first substrate 21, the second substrate 50, and a terminal part 40. The first substrate 21 is an insulating substrate and is formed into a strip shape using, for example, a film-like synthetic resin. The first substrate 21 is formed as a single substrate having a first end 21A and a second end 21B. The first substrate 21 is a deformable substrate provided with the first optical sensor 10A on the first end 21A side and the second optical sensor 10B on the second end 21B side.

The first substrate 21 is provided on the flexible printed circuit board 70 such that the first optical sensor 10A and the second optical sensor 10B are respectively positioned on the respective sides of the light source 60 in the circumferential direction 200C of the housing 200. The first substrate 21 has a notch 22 between the first optical sensor 10A and the second optical sensor 10B in the circumferential direction 200C of the housing 200. The notch 22 will be described later.

As illustrated in FIG. 4, the terminal part 40 is a member that electrically couples the first optical sensor 10A and the second optical sensor 10B of the first substrate 21 to the control circuit 122 and the power supply circuit 123 of the flexible printed circuit board 70. The terminal part 40 is provided on the first substrate 21 and is electrically coupled to a first wiring line 26A, a second wiring line 26B, third wiring lines 26C, and other components of the first substrate 21. The first wiring line 26A, the second wiring line 26B, and the third wiring lines 26C are metal lines in the same layer of the first substrate 21. The terminal part 40 supplies power signals (power) from the power supply circuit 123 to the first optical sensor 10A via the first wiring line 26A. The terminal part 40 supplies power signals (power) from the power supply circuit 123 to the second optical sensor 10B via the second wiring line 26B. The terminal part 40 includes a plurality of terminals and can be electrically coupled to a plurality of wiring lines.

The terminal part 40 is provided at the first end 21A of the first substrate 21. The terminal part 40 supplies power from the power supply circuit 123 to the first optical sensor 10A and the second optical sensor 10B.

Similarly to the first substrate 21, the second substrate 50 is an insulating substrate and is formed into a strip shape using, for example, a film-like synthetic resin. The second substrate 50 covers a sealing film 90 (refer to FIGS. 5 and 6) and is a deformable substrate.

In the present embodiment, as illustrated in FIG. 2, the flexible printed circuit board 70 is accommodated in the housing 200 such that the surface provided with the first optical sensor 10A, the second optical sensor 10B, and the light source 60 faces an inner peripheral surface 200B of the housing 200. If the flexible printed circuit board 70 has a light-transmitting property, the first optical sensor 10A, the second optical sensor 10B, and the light source 60 may be provided on the back surface opposite the front surface. In this case, the light source 60 only needs to be arranged so that light is emitted toward the flexible printed circuit board 70 and light transmitted through the flexible printed circuit board 70 is emitted toward the outside of the housing 200.

As illustrated in FIG. 2, the light source 60 is provided in the first housing 210 of the housing 200, and is capable of emitting light toward the finger Fg wearing the housing 200. The light source 60 is disposed in the notch 22 of the first substrate 21. For example, inorganic light-emitting diodes (LEDs) or organic electroluminescent (EL) diodes (organic light-emitting diodes (OLEDs)) are used as the light source 60. The light source 60 emits light having predetermined wavelengths. In the present embodiment, the light source 60 includes a plurality of light sources so as to be capable of emitting near-infrared light, red light, green light, or the like.

Light emitted from the light source 60 is reflected on the surface of a detection target, such as the finger Fg, and enters the first optical sensor 10A and the second optical sensor 10B. As a result, the detection device 1 can detect a fingerprint by detecting a shape of asperities on the surface of the finger Fg or the like. Alternatively, the light emitted from the light source 60 may be reflected in the finger Fg or the like or transmitted through the finger Fg or the like and enter the first optical sensor 10A and the second optical sensor 10B. Thus, the detection device 1 can detect information on a living body in the finger Fg or the like. Examples of the information on the living body include, but are not limited to, pulse waves, pulsation, and a vascular image of the finger or a palm. That is, the detection device 1 may be configured as a fingerprint detection device to detect the fingerprint or a vein detection device to detect a vascular pattern of, for example, veins.

The first optical sensor 10A and the second optical sensor 10B each detect light emitted by the light source 60 and reflected by the finger Fg or the like, light directly incident on the optical sensor, and other light. The first optical sensor 10A and the second optical sensor 10B are organic photodiodes (OPDs). The first optical sensor 10A is provided to the housing 200 so as to be adjacent to a first end 61 of the light source 60 in the circumferential direction 200C of the housing 200. The second optical sensor 10B is provided to the housing 200 so as to be adjacent to a second end 62 of the light source 60 in the circumferential direction 200C of the housing 200.

As illustrated in FIG. 4, the first optical sensor 10A and the second optical sensor 10B each include a plurality of photodiodes PD (refer to FIG. 4), which are organic photodiodes. The first optical sensor 10A includes three lower electrodes 11A, 11B, and 11C arranged along the circumferential direction 200C. The second optical sensor 10B includes three lower electrodes 11D, 11E, and 11F arranged along the circumferential direction 200C. The first optical sensor 10A and the second optical sensor 10B are provided on a single first substrate 21 and are electrically coupled to the flexible printed circuit board 70 via the first substrate 21.

In the following description, a first direction Dx is one direction in a plane parallel to the first substrate 21 and is the same direction as the circumferential direction 200C. A second direction Dy is one direction in the plane parallel to the first substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the first substrate 21. The term "plan view" refers to a positional relation when viewed from a direction perpendicular to the first substrate 21.

As illustrated in FIG. 4, the first optical sensor 10A has a multilayered configuration in which one upper electrode 15A covers three lower electrodes 11A, 11B, and 11C arranged in the first direction Dx. The second optical sensor 10B has a multilayered configuration in which one upper electrode 15B covers three lower electrodes 11D, 11E, and 11F arranged in the first direction Dx. An upper electrode 15 includes the upper electrode 15A of the first optical sensor 10A and the upper electrode 15B of the second optical sensor 10B. The upper electrode 15A covers the three lower electrodes 11A, 11B, and 11C in plan view. The upper electrode 15B covers the three lower electrodes 11D, 11E, and 11F in plan view. The upper electrode 15A and the upper electrode 15B have a rectangular surface and are independent electrodes that are not electrically coupled.

The first substrate 21 includes a first power supply electrode 25A and a second power supply electrode 25B extending along the second direction Dy. The first power supply electrode 25A is provided between the first end 21A of the first substrate 21 and the first optical sensor 10A in the first direction Dx. The second power supply electrode 25B is provided between the second end 21B of the first substrate 21 and the second optical sensor 10B in the first direction Dx. The first power supply electrode 25A is electrically coupled to the terminal part 40 of the first substrate 21 via the first wiring line 26A and supplied with power signals from the power supply circuit 123 (refer to FIG. 3) via the terminal part 40. The second power supply electrode 25B is electrically coupled to the terminal part 40 of the first substrate 21 via the second wiring line 26B and supplied with power signals from the power supply circuit 123 via the terminal part 40.

Each of the lower electrodes 11A, 11B, and 11C of the first optical sensor 10A is coupled to the terminal part 40 via the third wiring line 26C. The lower electrode 11F of the second optical sensor 10B is coupled to the terminal part 40 via the third wiring line 26C. The third wiring lines 26C of the first substrate 21 are coupled to a detection circuit 48 of the control circuit 122 via the terminal part 40 and signal lines SL of the flexible printed circuit board 70. In other words, the detection circuit 48 is electrically coupled to the lower electrodes 11A, 11B, and 11C of the first optical sensor 10A and the lower electrode 11F of the second optical sensor 10B via the signal lines SL. The detection circuit 48 of the control circuit 122 and the power supply circuit 123 may be formed as individual circuits.

The third wiring lines 26C are formed, for example, of metal lines, and are formed of a material having better conductivity than the lower electrodes 11 of the photodiodes PD. The third wiring lines 26C are formed, for example, of a light-transmitting conductive material, such as indium tin oxide (ITO). The third wiring lines 26C may be formed, for example, in the same layer as the lower electrodes 11, and/or may be formed of metal.

The upper electrode 15A of the first optical sensor 10A is coupled to the first power supply electrode 25A via a conductive member 24 and electrically coupled to the terminal part 40 via the first wiring line 26A coupled to the first power supply electrode 25A. The upper electrode 15B of the second optical sensor 10B is coupled to the second power supply electrode 25B via a conductive member 24 and electrically coupled to the terminal part 40 via the second wiring line 26B coupled to the second power supply electrode 25B. As a result, the upper electrode 15A and the upper electrode 15B are supplied with power from the independent power systems of the first power supply electrode 25A and the second power supply electrode 25B, respectively. Each of the conductive members 24 is formed of a conductive material and covers the entire surface of the first power supply electrode 25A or the second power supply electrode 25B. The conductive member 24 electrically couples the first power supply electrode 25A to the upper electrode 15A or the second power supply electrode 25B to the upper electrode 15B. The upper electrode 15A and the upper electrode 15B may be directly coupled to the first power supply electrode 25A and the second power supply electrode 25B, respectively, without the conductive member 24.

As illustrated in FIG. 4, the first substrate 21 is a single integrally formed common substrate having the first end 21A provided with the first optical sensor 10A and the second end 21B provided with the second optical sensor 10B. The first substrate 21 has the notch 22 formed between the first end 21A provided with the first optical sensor 10A and the second end 21B provided with the second optical sensor 10B in the first direction Dx. The first substrate 21 has the notch 22 between the first optical sensor 10A and the second optical sensor 10B, and a connecting portion 23 in contact with the notch 22 and positioned between the first optical sensor 10A and the second optical sensor 10B.

The notch 22 is formed to have a length in the first direction Dx longer than the length of the light source 60. The notch 22 is formed to have a length in the second direction Dy longer than the length of the light source 60 and shorter than the length (width) of the first substrate 21. The first substrate 21 is integrally formed by connecting the regions of the first optical sensor 10A and the second optical sensor 10B with the connecting portion 23 adjacent to the notch 22. The notch 22 is formed in such a shape that allows the light source 60 to be disposed therein. While the notch 22 according to the present embodiment is formed in a substantially rectangular shape in plan view, it may have another shape, such as a semicircle, a triangle, or a polygon. The connecting portion 23 is provided with the second wiring line 26B and the third wiring line 26C.

The control circuit 122 is a circuit that supplies control signals to the photodiodes PD to control detection operations thereof. Each of the photodiodes PD outputs an electrical signal corresponding to the light emitted thereto as a detection signal to the detection circuit 48. The detection signals from the photodiodes PD according to the present embodiment are sequentially output to the detection circuit 48 in a time-division manner. In other words, the signal lines SL are sequentially electrically coupled to the detection circuit 48 in a time-division manner. Thus, the detection device 1 detects information on the detection target based on the detection signals from the photodiodes PD.

The first power supply electrode 25A and the second power supply electrode 25B are supplied with power signals from the power supply circuit 123 via the terminal part 40 and supply power signals to the upper electrode 15A and the upper electrode 15B. In the example illustrated in FIG. 4, the first power supply electrode 25A and the second power supply electrode 25B are formed in a substantially rectangular shape extending in the second direction Dy in plan view and have the same area (size).

The power supply circuit 123 supplies a first reference potential Vref (refer to FIG. 7) to the lower electrodes 11 of the photodiodes PD via the terminal part 40, and supplies a second reference potential Vorg (refer to FIG. 7) to the upper electrodes 15 of the photodiodes PD. The first reference potential Vref is a predetermined fixed potential. The second reference potential Vorg is a predetermined fixed potential.

The first reference potential Vref has a higher potential than the second reference potential Vorg. As a result, the photodiode PD is driven into a reverse bias state.

Each of the photodiodes PD outputs an electrical signal (photocurrent Id) corresponding to light emitted thereto to the detection circuit 48. Thus, the detection device 1 detects information on the detection target based on the photocurrents Id from the photodiodes PD.

The lower electrodes 11 of the first optical sensor 10A and the lower electrodes 11 of the second optical sensor 10B are electrically coupled to each other and are also electrically coupled to the terminal part 40. The lower electrodes 11B and 11E according to the present embodiment are electrically coupled to each other via a fourth wiring line 26D and are also electrically coupled to the terminal part 40. The lower electrodes 11C and 11D are electrically coupled to each other via a fourth wiring line 26E and are also electrically coupled to the terminal part 40.

Similarly to the third wiring lines 26C, the fourth wiring lines 26D and 26E are formed, for example, of metal lines and are formed of a material having better conductivity than the lower electrodes 11 of the photodiodes PD.

The first optical sensor 10A includes a plurality of first optical sensors 10A1, 10A2, and 10A3; and the second optical sensor 10B includes a plurality of second optical sensors 10B1, 10B2, and 10B3. Out of the first optical sensors 10A1, 10A2, and 10A3 and the second optical sensors 10B1, 10B2, and 10B3, a pair of the first optical sensor 10A and the second optical sensor 10B located at the same ordinal position as counted from the notch 22 is defined as a combination of the first optical sensor 10A and the second optical sensor 10B. In at least one combination of the first optical sensor 10A and the second optical sensor 10B, the respective lower electrodes 11 are electrically coupled to each other.

The combinations of the first optical sensor 10A and the second optical sensor 10B according to the present embodiment are: the first optical sensor 10A1 and the second optical sensor 10B1; the first optical sensor 10A2 and the second optical sensor 10B2; and the first optical sensor 10A3 and the second optical sensor 10B3. In the combination of the first optical sensor 10A2 and the second optical sensor 10B2, the lower electrodes 11B and 11E located at the second position as counted from the notch 22 are electrically coupled to each other. In the combination of the first optical sensor 10A3 and the second optical sensor 10B3, the lower electrodes 11C and 11D located at the first position as counted from the notch 22 are electrically coupled to each other.

This configuration can reduce variations in the characteristics of pulse wave signals that would be caused due to the difference in distance from the light source 60 to the lower electrode 11.

In the other combination of the first optical sensor 10A and the second optical sensor 10B, the respective lower electrodes 11 are not electrically coupled to each other. The lower electrodes 11A and 11F according to the present embodiment are not electrically coupled to each other.

In the combination of the first optical sensor 10A and the second optical sensor 10B disposed closest to the notch 22, the respective lower electrodes 11 are electrically coupled to each other. In the combination of the first optical sensor 10A and the second optical sensor 10B disposed farthest from the notch 22, the respective lower electrodes 11 are not electrically coupled to each other. In the present embodiment, in the combination of the first optical sensor 10A3 and the second optical sensor 10B3 disposed closest to the notch 22, the lower electrodes 11C and 11D are electrically coupled to each other. In the combination of the first optical sensor 10A1 and the second optical sensor 10B1 disposed farthest from the notch 22, the lower electrodes 11A and 11F are not electrically coupled to each other.

This configuration can prevent the lower electrodes 11A and 11F disposed farthest from the light source 60 from receiving different pulse wave signals.

In at least one combination of the first optical sensor 10A and the second optical sensor 10B, the areas of the respective lower electrodes 11 are equivalent. In the present embodiment, in each of the combinations of the lower electrodes 11A and 11F, the lower electrodes 11B and 11E, and the lower electrodes 11C and 11D, the areas of the lower electrodes 11 are equivalent.

As illustrated in FIG. 5, the first optical sensor 10A includes the first substrate 21 (first end 21A), the photodiode PD, and the second substrate 50 facing the first substrate 21. The first optical sensor 10A according to the present embodiment further includes a first insulating layer 27, an insulating film 28, a sealing film 90, and a second insulating layer 270.

The first insulating layer 27 is provided on the upper side of the first substrate 21. The first insulating layer 27 is disposed between the first substrate 21 and the photodiode PD. The second insulating layer 270 is provided on the upper side of the insulating film 28. The second insulating layer 270 is disposed between the second substrate 50 and the photodiode PD. The first insulating layer 27 and the second insulating layer 270 may be inorganic insulating films or organic insulating films.

The insulating film 28 is provided on the upper side of the first insulating layer 27. The insulating film 28 is disposed between the first insulating layer 27 and the photodiode PD. Similarly to the first insulating layer 27 and the second insulating layer 270, the insulating film 28 may be an inorganic insulating film or an organic insulating film.

The photodiode PD is provided on the upper side of the first insulating layer 27 as a sensor element. The photodiode PD includes the lower electrode 11, a lower buffer layer 12, an active layer 13, an upper buffer layer 14, and the upper electrode 15 (15A). In the photodiode PD, the lower electrode 11, the lower buffer layer 12 (hole transport layer), the active layer 13, the upper buffer layer 14 (electron transport layer), and the upper electrode 15 are stacked in this order in the third direction Dz perpendicular to the first substrate 21. When the upper electrode 15 is not provided, the upper buffer layer 14 serves as a cathode electrode of the photodiode PD.

The lower electrode 11 is an anode electrode of the photodiode PD, and is formed, for example, of a light-transmitting conductive material, such as indium tin oxide (ITO). The active layer 13 changes in characteristics (for example, voltage-current characteristics and resistance value) depending on light emitted thereto. An organic material is used as a material of the active layer 13. Specifically, the active layer 13 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 13, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 13 can be formed by a vapor deposition process (dry process) using any of the low-molecular-weight organic materials listed above. In this case, the active layer 13 may be, for example, a multilayered film of CuPc and F₁₆CuPc, or a multilayered film of rubrene and C₆₀. The active layer 13 can also be formed by a coating process (wet process). In this case, the active layer 13 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 13 can be a film made of a mixture of P3HT and PCBM, or a film made of a mixture of F8BT and PDI.

The lower buffer layer 12 is a hole transport layer. The upper buffer layer 14 is an electron transport layer. The lower buffer layer 12 and the upper buffer layer 14 are provided to facilitate holes and electrons generated in the active layer 13 to reach the lower electrode 11 or the upper electrode 15. The lower buffer layer 12 (hole transport layer) is in direct contact with the top of the lower electrode 11 and is also provided in an area between the adjacent lower electrodes 11. The active layer 13 is in direct contact with the top of the lower buffer layer 12. The material of the hole transport layer is a metal oxide layer. For example, tungsten oxide (WO₃) or molybdenum oxide is used as the metal oxide layer.

The upper buffer layer 14 (electron transport layer) is in direct contact with the top of the active layer 13, and the upper electrode 15 is in direct contact with the top of the upper buffer layer 14. Polyethylenimine ethoxylated (PEIE) is used as a material of the electron transport layer.

The materials and the manufacturing methods of the lower buffer layer 12, the active layer 13, and the upper buffer layer 14 are merely exemplary, and other materials and manufacturing methods may be used. For example, each of the lower buffer layer 12 and the upper buffer layer 14 is not limited to a single-layer film, and may be formed as a multilayered film that includes an electron blocking layer and a hole blocking layer.

The upper electrode 15 is provided on the upper buffer layer 14. The upper electrode 15 is a cathode electrode of the photodiode PD, and is continuously formed over the entire first optical sensor 10A and the entire second optical sensor 10B. In other words, the upper electrode 15 is continuously provided on a plurality of photodiodes PD. The upper electrode 15 faces the lower electrodes 11 (11A, 11B, and 11C) with the lower buffer layer 12, the active layer 13, and the upper buffer layer 14 interposed therebetween. The upper electrode 15 is formed, for example, of a light-transmitting conductive material such as ITO or indium zinc oxide (IZO). A portion of an edge region of an upper surface 150 of the upper electrode 15 is electrically coupled to the conductive member 24. The conductive member 24 is electrically coupled to the first power supply electrode 25A and supplies the power signals from the first power supply electrode 25A to the upper electrode 15. In the first optical sensor 10A, the sealing film 90 is provided on the upper electrode 15, the conductive member 24, and other components to satisfactorily seal the photodiode PD. The upper electrode 15 may be a multilayered film of a plurality of light-transmitting conductive materials.

A sealing film 90 is provided on the upper electrode 15. The sealing film 90 is an inorganic film, such as a silicon nitride film and an aluminum oxide film, or a resin film made of acrylic. The sealing film 90 is not limited to a single layer and may be a multilayered film composed of two or more layers obtained by combining the inorganic film and the resin film described above. The sealing film 90 satisfactorily seals the photodiode PD and inhibits moisture from entering from the upper surface side.

As illustrated in FIG. 6, the second optical sensor 10B includes the three lower electrodes 11 (11D, 11E, and 11F) of the second optical sensor 10B in a different region of the first substrate 21 from the lower electrodes 11 (11A, 11B, and 11C) of the first optical sensor 10A. The lower electrodes 11D, 11E, and 11F are covered by the lower buffer layer 12, the active layer 13, the upper buffer layer 14, and the upper electrode 15 (15B). The second optical sensor 10B according to the present embodiment includes the first substrate 21 (second end 21B), the photodiode PD, the first insulating layer 27, the insulating film 28, the second substrate 50 facing the first substrate 21, and the second insulating layer 270. The photodiode PD, the first insulating layer 27, the insulating film 28, and the second insulating layer 270 have the same configuration as the photodiode PD, the first insulating layer 27, the insulating film 28, and the second insulating layer 270 of the first optical sensor 10A described above. In other words, the photodiode PD of the second optical sensor 10B includes the lower electrodes 11 (11D, 11E, and 11F), the lower buffer layer 12, the active layer 13, the upper buffer layer 14, and the upper electrode 15 (15B). The first optical sensor 10A and the second optical sensor 10B according to the present embodiment are organic photodiodes.

In the second optical sensor 10B, a portion of an end of the upper surface 150 of the upper electrode 15 (15B) is electrically coupled to the conductive member 24, and the conductive member 24 is electrically coupled to the second power supply electrode 25B. The second optical sensor 10B supplies the power signals from the second power supply electrode 25B to the upper electrode 15. In the second optical sensor 10B, the sealing film 90 is provided on the upper electrode 15 (15B), the conductive member 24, and other components to satisfactorily seal the photodiode PD.

FIG. 7 is a circuit diagram illustrating a configuration example of the detection circuit according to the embodiment. As illustrated in FIG. 7, a plurality of photodiodes PD1, PD2, PD3, and PD4 are coupled to the detection circuit 48 via coupling switches SSW1, SSW2, SSW3, and SSW4, respectively.

In the following description, the photodiodes PD1, PD2, PD3, and PD4 will each be simply referred to as the photodiode PD when they need not be distinguished from one another. The coupling switches SSW1, SSW2, SSW3, and SSW4 will each be simply referred to as the coupling switch SSW when they need not be distinguished from one another.

The photodiode PD1 is a photodiode including the lower electrode 11A. The photodiode PD2 is a photodiode including the lower electrodes 11B and 11E. The photodiode PD3 is a photodiode including the lower electrodes 11C and 11D. The photodiode PD4 is a photodiode including the lower electrode 11F.

The control circuit 122 includes the detection circuit 48 and a storage circuit 49. The detection circuit 48 includes a detection signal amplifying circuit 45 and an analog-to-digital converter (ADC) circuit 47.

The detection circuit 48 is a current detection circuit that measures the photocurrent Id output from the photodiode PD. The detection circuit 48 is a signal processing circuit having functions of at least the detection signal amplifying circuit 45 and the ADC circuit 47.

The storage circuit 49 temporarily stores therein information on the photocurrent Id measured by the detection circuit 48. The storage circuit 49 may be, for example, a random-access memory (RAM) or a register circuit.

The anode of each photodiode PD is supplied with the second reference potential Vorg from the power supply circuit 123. In other words, each of the upper electrodes 15 of the first optical sensor 10A and the second optical sensor 10B is supplied with the second reference potential Vorg. The cathode of the photodiode PD is coupled to the detection circuit 48 via the coupling switch SSW.

A sensor capacitance Cd is coupled in parallel with the photodiode PD. The sensor capacitance Cd is a capacitance formed between the upper electrode 15 and the lower electrode 11 of the photodiode PD.

An inverting input (–) of the detection signal amplifying circuit 45 is coupled to the photodiode PD via the coupling switch SSW. The photodiodes PD1, PD2, PD3, and PD4 are coupled in parallel to the inverting inputs (–) of the detection signal amplifying circuits 45 via the coupling switches SSW1, SSW2, SSW3, and SSW4, respectively.

A non-inverting input (+) of the detection signal amplifying circuit 45 is supplied with the first reference potential Vref from the power supply circuit 123. The output of the detection signal amplifying circuit 45 is coupled to the ADC circuit 47.

The detection signal amplifying circuit 45 amplifies the detection signals from the photodiodes. The ADC circuit 47 converts analog signals output from the detection signal amplifying circuit 45 into digital signals. The detection circuit 48 measures the photocurrent Id output from the photodiode PD, performs signal processing, such as A/D conversion, and stores a sensor value Raw, which is a detection value of each photodiode PD corresponding to the photocurrent Id, in the storage circuit 49.

The detection circuit 48 acquires the sensor value Raw(n) of each photodiode PD (where n is an integer from 1 to N, and N is the total number of frames). The control circuit 122 outputs an output value Data(n) of each photodiode PD (where n is an integer from 1 to N, and N is the total number of frames) to an external host (not illustrated).

FIG. 8 is a configuration diagram illustrating a configuration example of a first optical sensor and a second optical sensor according to a comparative example. As illustrated in FIG. 8, a detection device 1A according to the comparative example is different from the detection device 1 according to the embodiment in that the lower electrodes 11 arranged symmetrically at equal distances on the respective sides of the notch 22 in the first direction Dx are not electrically coupled to each other.

As a result, the total light-receiving area of the optical sensor decreases, which may result in lower light-receiving sensitivity of the optical sensor.

By contrast, in the detection device 1 according to the first embodiment, the lower electrodes 11 arranged symmetrically at equal distances on the respective sides of the notch 22 in the first direction Dx are electrically coupled to each other. As a result, the light-receiving area of the photodiodes PD increases, thereby improving the light-receiving sensitivity of the optical sensor.

The detection device 1A according to the comparative example is different from the detection device 1 according to the embodiment in that the area of each lower electrode 11 of the first optical sensor 10A and the area of each lower electrode 11 of the second optical sensor 10B are equivalent and uniform.

With this configuration, as the distance from the light source 60 to the lower electrode 11 increases, the amount of light reaching the optical sensor decreases exponentially, which may result in lower light-receiving sensitivity of the optical sensor.

By contrast, in the detection device 1 according to the first embodiment, the area of the lower electrode 11 of the first optical sensor 10A or the area of the lower electrode 11 of the second optical sensor 10B in at least one combination of the first optical sensor 10A and the second optical sensor 10B is different from the area of the lower electrode 11 in another combination of the first optical sensor 10A and the second optical sensor 10B. In the present embodiment, the area of each of the lower electrodes 11A and 11F in the combination of the first optical sensor 10A1 and the second optical sensor 10B1 is different from that of each of the lower electrodes 11C and 11D in the combination of the first optical sensor 10A3 and the second optical sensor 10B3. The area of each of the lower electrodes 11A and 11F in the combination of the first optical sensor 10A1 and the second optical sensor 10B1 is different from that of each of the lower electrodes 11B and 11E in the combination of the first optical sensor 10A2 and the second optical sensor 10B2.

The area of the lower electrode 11 in the combination of the first optical sensor 10A and the second optical sensor 10B disposed farthest from the notch 22 is larger than the area of the lower electrode 11 in the combination of the first optical sensor 10A and the second optical sensor 10B disposed closest to the notch 22. In other words, the area of each of the lower electrodes 11A and 11F in the combination of the first optical sensor 10A1 and the second optical sensor 10B1 disposed farthest from the notch 22 is larger than the area of each of the lower electrodes 11C and 11D in the combination of the first optical sensor 10A3 and the second optical sensor 10B3 disposed closest to the notch 22.

This configuration can increase the light-receiving sensitivity and inhibit the amount of light reaching the optical sensor from decreasing exponentially as the distance from the light source 60 to the lower electrode 11 increases.

### SECOND EMBODIMENT

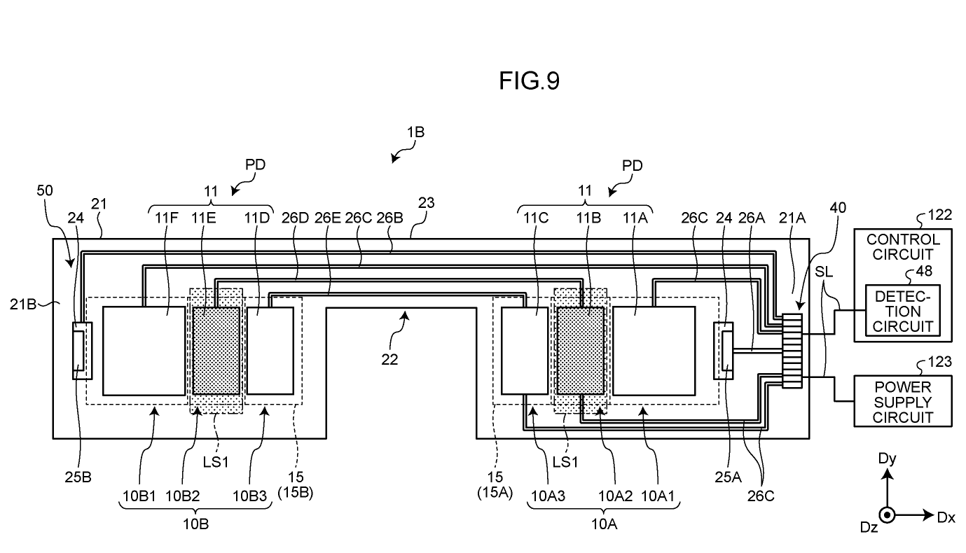

FIG. 9 is a configuration diagram illustrating a configuration example of a first optical sensor and a second optical sensor according to a second embodiment. In the following description, the same components as those described in the embodiment described above are denoted by the same reference numerals, and the description thereof will not be repeated.

In a detection device 1B according to the second embodiment, the first optical sensor 10A and the second optical sensor 10B further include a light-shielding layer LS1, and the light-shielding layer LS1 is larger than the lower electrode 11. At least one lower electrode 11 is covered by the light-shielding layer LS1 in plan view and is electrically coupled to the terminal part 40.

As illustrated in FIG. 9, the light-shielding layer LS1 is provided at a position overlapping the lower electrode 11B in the third direction Dz, and the light-shielding layer LS1 is provided at a position overlapping the lower electrode 11E in the third direction Dz. Each of the light-shielding layers LS1 is formed of a non-light-transmitting material.

The light-shielding layer LS1 according to the present embodiment is larger than the lower electrodes 11B and 11E. Each of the lower electrodes 11B and 11E is covered by the light-shielding layer LS1 in plan view, and the lower electrode 11B is electrically coupled to the terminal part 40.

In at least one combination of the first optical sensor 10A and the second optical sensor 10B, the respective lower electrodes 11 covered by the light-shielding layers LS1 are electrically coupled to each other. In the present embodiment, in the combination of the first optical sensor 10A2 and the second optical sensor 10B2, the lower electrodes 11B and 11E covered by the respective light-shielding layers LS1 are electrically coupled to each other.

In this case, the output value Data(n) of the photodiode PD1 is the difference obtained by subtracting the sensor value Raw(n) of the photodiode PD2 from the sensor value Raw(n) of the photodiode PD1.

Similarly to the output value Data(n) of the photodiode PD1, the output value Data(n) of the photodiode PD3 is the difference obtained by subtracting the sensor value Raw(n) of the photodiode PD2 from the sensor value Raw(n) of the photodiode PD3.

Similarly to the output value Data(n) of the photodiode PD1, the output value Data(n) of the photodiode PD4 is the difference obtained by subtracting the sensor value Raw(n) of the photodiode PD2 from the sensor value Raw(n) of the photodiode PD4.

Thus, using the photodiode PD2 shielded by the light-shielding layer LS1 as a reference element can cancel noise in the optical sensor 10 and reduce electromagnetic noise.

The area of each of the lower electrodes 11 covered by the light-shielding layer LS1 is equivalent to the area of the lower electrode 11 of the first optical sensor 10A or the second optical sensor 10B, wherein the lower electrodes 11 of the first optical sensor 10A and the second optical sensor 10B are not covered by the light-shielding layer LS1 and are electrically coupled to each other. In the present embodiment, the area of each of the lower electrodes 11B and 11E covered by the light-shielding layer LS1 is equivalent to that of the lower electrode 11C of the first optical sensor 10A3 or the lower electrode 11D of the second optical sensor 10B3, wherein the lower electrodes 11 of the first optical sensor 10A3 and the second optical sensor 10B3 are not covered by the light-shielding layer LS1 and are electrically coupled to each other.

Thus, the light-receiving area of the unshielded photodiode PD3 is equivalent to that of the shielded photodiode PD2, thereby canceling the noise components in the photodiode PD3 and eliminating the need for gain adjustment.

The total area of the lower electrodes 11 covered by the light-shielding layer LS1 is equivalent to the area of the lower electrode 11 of the first optical sensor 10A or the second optical sensor 10B, wherein the lower electrodes 11 of the first optical sensor 10A and the second optical sensor 10B are not electrically coupled to each other. In the present embodiment, the total area of the lower electrodes 11B and 11E covered by the light-shielding layer LS1 is equivalent to the area of the lower electrode 11A of the first optical sensor 10A1 or the lower electrode 11F of the second optical sensor 10B1, wherein the lower electrodes 11 of the first optical sensor 10A1 and the second optical sensor 10B1 are not electrically coupled to each other.

Thus, the light-receiving area of each of the unshielded photodiodes PD1 and PD4 is equivalent to that of the shielded photodiode PD2, thereby canceling the noise components in the photodiodes PD1 and PD4 and eliminating the need for gain adjustment.

In the combination of the first optical sensor 10A and the second optical sensor 10B other than the combination of the first optical sensor 10A and the second optical sensor 10B disposed closest to the notch 22, the respective lower electrodes 11 are covered by the light-shielding layer LS1 in plan view. In the present embodiment, in the combination of the first optical sensor 10A2 and the second optical sensor 10B2 other than the combination of the first optical sensor 10A3 and the second optical sensor 10B3 disposed closest to the notch 22, the lower electrodes 11B and 11E are covered by the light-shielding layer LS1 in plan view.

With this configuration, the photodiodes PD closest to the light source 60 can be effectively used for optical sensing because they receive a large amount of green light and the like.

In the combination of the first optical sensor 10A and the second optical sensor 10B other than the combination of the first optical sensor 10A and the second optical sensor 10B disposed farthest from the notch 22, the respective lower electrodes 11 are covered by the light-shielding layer LS1 in plan view. In the present embodiment, in the combination of the first optical sensor 10A2 and the second optical sensor 10B2 other than the combination of the first optical sensor 10A1 and the second optical sensor 10B1 disposed farthest from the notch 22, the lower electrodes 11B and 11E are covered by the light-shielding layer LS1 in plan view.

With this configuration, the photodiodes PD farthest from the light source 60 can improve the quality of detection signals for near-infrared light, red light, and the like.

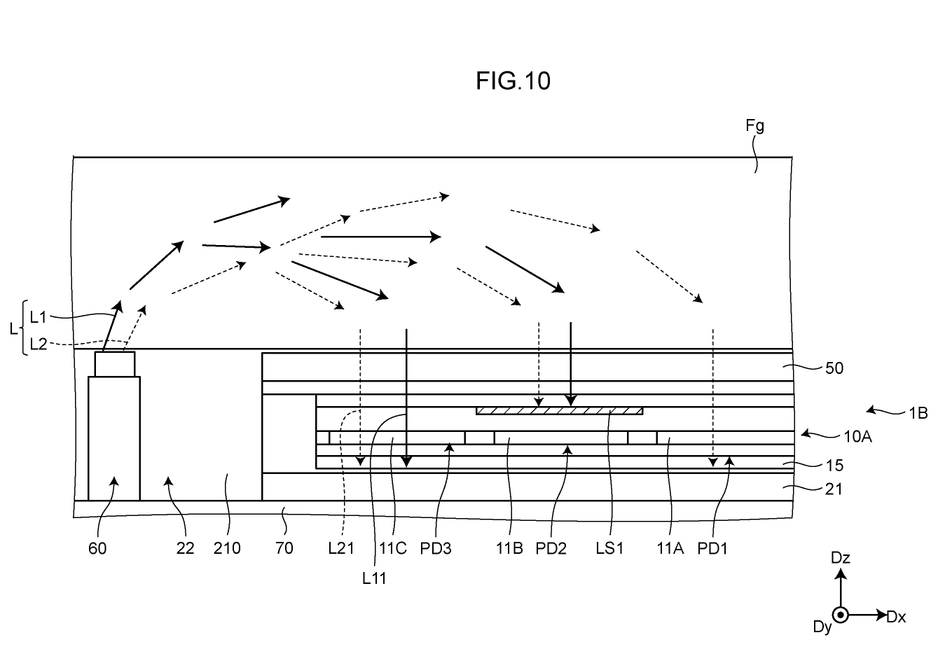

FIG. 10 is a schematic sectional view illustrating a configuration example of a detection device according to the second embodiment. FIG. 10 is a schematic sectional view illustrating a configuration example of the optical sensor taken along X-X' illustrated in FIG. 3. As illustrated in FIG. 10, when the light source 60 of the detection device 1B worn on the finger Fg emits light L, the light L passes through the housing 200 and enters the finger Fg. The light L includes green light L1 and near-infrared light L2. The near-infrared light L2 may be red light.

The light L enters the finger Fg through the skin and is transmitted through or reflected by muscle tissue, arteries, veins, and the like. Reflected light L11 and L21 resulting from reflection of the green light L1 and the near-infrared light L2 in the finger Fg travels toward the detection device 1B. When the optical sensor 10 of the detection device 1B is positioned in a direction in which the reflected light L11 and L21 is output toward the outside of the finger Fg, the photodiodes PD of the detection device 1B receive the reflected light L11 and L21.

The lower electrode 11B positioned between the lower electrode 11A disposed farthest from the light source 60 and the lower electrode 11C disposed closest to the light source 60 is shielded from the reflected light L11 and L21 by the light-shielding layer LS1.

The lower electrode 11C is used as an optical sensing element for the green light L1, and the lower electrode 11A is used as an optical sensing element for the near-infrared light L2.

Since both the lower electrode 11A and the lower electrode 11C are close to the shielded lower electrode 11B, the light detection accuracy of the photodiodes PD1 and PD3 can be improved. While FIG. 10 illustrates a configuration example of the first optical sensor 10A, this configuration can also improve the light detection accuracy of the photodiodes PD3 and PD4 in the second optical sensor 10B.

Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection device comprising:
a first substrate having a notch between both ends in a first direction;
a light source disposed in the notch of the first substrate;
a terminal part provided at one end of the first substrate in the first direction;
at least one first optical sensor provided to the first substrate; and
at least one second optical sensor provided to the first substrate, wherein
the first optical sensor and the second optical sensor are disposed so as to sandwich the notch of the first substrate in the first direction,
the first optical sensor and the second optical sensor each comprise a lower electrode, a lower buffer layer, and an active layer,
the first optical sensor and the second optical sensor each comprise an upper electrode provided on the active layer and supplied with a reference potential from the terminal part, and
the lower electrode of the first optical sensor and the lower electrode of the second optical sensor are electrically coupled to each other and are electrically coupled to the terminal part.

2. The detection device according to claim 1, further comprising:
a plurality of the first optical sensors and a plurality of the second optical sensors, wherein
pairs of the first optical sensor and the second optical sensor located at the same ordinal position as counted from the notch out of the first optical sensors and the second optical sensors are defined as combinations of the first optical sensor and the second optical sensor, and
in at least one of the combinations of the first optical sensor and the second optical sensor, the respective lower electrodes are electrically coupled to each other.

3. The detection device according to claim 2, wherein, in another combination of the first optical sensor and the second optical sensor, the respective lower electrodes are not electrically coupled to each other.

4. The detection device according to claim 2, wherein
in the combination of the first optical sensor and the second optical sensor disposed closest to the notch, the respective lower electrodes are electrically coupled to each other, and
in the combination of the first optical sensor and the second optical sensor disposed farthest from the notch, the respective lower electrodes are not electrically coupled to each other.

5. The detection device according to claim 2, further comprising:
a light-shielding layer, wherein
the light-shielding layer is larger than the lower electrode, and
at least one of the lower electrodes is covered by the light-shielding layer in plan view and is electrically coupled to the terminal part.

6. The detection device according to claim 5, wherein, in at least one of the combinations of the first optical sensor and the second optical sensor, the respective lower electrodes covered by the light-shielding layer are electrically coupled to each other.

7. The detection device according to claim 6, wherein the area of each of the lower electrodes covered by the light-shielding layer is equivalent to the area of the lower electrode of the first optical sensor or the second optical sensor, where the lower electrodes of the first optical sensor and the second optical sensor are not covered by the light-shielding layer and are electrically coupled to each other.

8. The detection device according to claim 6, wherein the total area of the lower electrodes covered by the light-shielding layer is equivalent to the area of the lower electrode of the first optical sensor or the second optical sensor, where the lower electrodes of the first optical sensor and the second optical sensor are not electrically coupled to each other.

9. The detection device according to claim 5, wherein, in a combination of the first optical sensor and the second optical sensor other than the combination of the first optical sensor and the second optical sensor disposed closest to the notch, the respective lower electrodes are covered by the light-shielding layer in plan view.

10. The detection device according to claim 5, wherein, in a combination of the first optical sensor and the second optical sensor other than the combination of the first optical sensor and the second optical sensor disposed farthest from the notch, the respective lower electrodes are covered by the light-shielding layer in plan view.

11. The detection device according to claim 2, wherein, in the at least one of the combinations of the first optical sensor and the second optical sensor, the areas of the respective lower electrodes are equivalent.

12. The detection device according to claim 11, wherein the area of the lower electrode of the first optical sensor or the area of the lower electrode of the second optical sensor in the at least one of the combinations of the first optical sensor and the second optical sensor is different from each of the areas of the lower electrodes in another combination of the first optical sensor and the second optical sensor.

13. The detection device according to claim 2, wherein the area of each of the lower electrodes in the combination of the first optical sensor and the second optical sensor disposed farthest from the notch is larger than the area of each the lower electrodes in the combination of the first optical sensor and the second optical sensor disposed closest to the notch.

14. The detection device according to claim 1, wherein the
first optical sensor and the second optical sensor are organic photodiodes.
